(19) United States
(12) Patent Application Publication
(10) Pub. No.: US 2014/0270076 A1
(43) Pub. Date: Sep. 18, 2014

(54) Rodent Ionizing Radiation Treatment Device

(71) Applicant: THE OHIO STATE UNIVERSITY, Columbus, OH (US)

(72) Inventors: Christopher E. Pelloski, Columbus, OH (US); James L. Sommerfeld, Westerville, OH (US); Kathryn Bondra, Blacklick, OH (US); Lanchun Lu, Powell, OH (US)

(73) Assignee: THE OHIO STATE UNIVERSITY, Columbus, OH (US)

(21) Appl. No.: 13/834,025

(22) Filed: Mar. 15, 2013

(51) Int. Cl. A61N 5/10

(52) U.S. Cl. CPC A61N 5/1031
USPC 378/65

(57) ABSTRACT

The present application relates to rodent radiation device that enables simultaneous radiation treatment of a plurality of small animals such as mice with localized radiation therapy. The device can provide clinically-relevant homogeneous radiation dosing and scheduling to a plurality of small animals or other in vivo cancer models simultaneously. Specifically, the multi station/unit design of the device allows for rapid loading of anesthetized small animals into radiation shield chambers that absorbs over 95% of the delivered dose and protects the remainder of the untreated animal, allowing for more clinically accurate recapitulation of radiotherapy regimens. In one embodiment, a total of 40-50 Gy is delivered in 20-25 fractions to small animals with more than 95% survival rates.

### TECHNICAL FIELD

The present application relates to ionizing radiation treatment devices that can be used to have homogenous radiation applied to a plurality of small animals such as rodents simultaneously. The present applications also relates to methods and protocols of using the radiation treatment device to deliver clinically relevant radiation dosing to the animals.

### BACKGROUND

Radiation therapy technique is not only used for cancer treatments but also applied in biological life science and cancer research. In radiation therapy experiments, cells or small animals (i.e. mice) are usually irradiated with radiation beams to study the mechanism of cell killing, the cell response to radiation, or the cancer treatment efficacy of chemo-radiation therapy. Historically, radiobiology research has been removed from the clinical experience. The cells of traditional radiobiology assays (notably, the clonogenic survival assay) are grown in tissue culture dishes. The in vitro passaging artifact results in cells that bear little molecular resemblance of the cells from whence they came. Early reports in fact indicate that the molecular changes from conventional fractionation and large-single doses have almost divergent molecular radiobiology and response characteristics as discussed in publications including: Tsai et al. Gene expression profiling of breast, prostate, and glioma cells following single versus fractionated doses of radiation. Cancer Res 2007:67(8):3845-3852; Hamilton et al. Conventional radiotherapy or hypofractionation? A study of molecular changes resulting from different radiation fractionation schemes. Cancer Biol Ther 2009:8(9):774-776 and Zelefsky et al. Tumor Control Outcomes After Hypofractionated and Single-Dose Stereotactic Image-Guided Intensity-Modulated Radiotherapy for Extracranial Metastases From Renal Cell Carcinoma. Int J Radiat Oncol Biol Phys 2011.

The use of animal-based tumors in general is believed to better approximate the clinical and biologic conditions than petri-dish/tissue culture models. Delivering clinically-relevant radiation dosing and scheduling to large numbers of small animals or other in vivo models however, has proven to be technically challenging. Investigators have attempted to circumvent these difficulties by either using very few numbers of experimental subjects, or delivering large, single-dosed fractions, oftentimes to the entire animal, as compared to most radiotherapy regimens that require multiple focal doses. Currently, there is no device available that allows for rapid radiation treatment of numerous small animals with focal dosing. Most groups abandon the animal model testing of radiation treatment due to the lack of suitable instrumentation.

### SUMMARY

The present application relates to a rodent ionizing radiation treatment device that comprises a plurality of rodent stations each including a shielding chamber that houses and shields a portion of a rodent from a projected ionizing radiation beam while leaving a target portion of each housed rodent exposed to the projected radiation beam such that the exposed target portion of each housed rodent is positioned within the radiation beam approximately equidistant from the center of the projected radiation beam at a predetermined distance from the beam source. Each exposed target portion of the rodent receives about same amount of radiation, with a homogeneous beam and dose distribution. In some embodiments, the shielding chambers are positioned about the periphery of the projected radiation beam. In some embodiments, the longitudinal dimension of the chambers is positioned perpendicularly to the periphery of the projected radiation beam and the open end of each chamber is directed inside the periphery towards the center of the beam. The shielding chambers deflect and scatter radiation from the projected radiation beam to enable homogeneous beam quality and radiation dose being applied to the stations. In some embodiments, the variance of the radiation experienced at the exposed target portion of each housed rodent is less than 30% of each other, in other embodiments, less than 20%, in additional embodiments, less than 15%, in further embodiments less than 10%, and in some embodiments, less than 5%.

The present application further relates to a method of irradiating target portions of a plurality of rodents simultaneously. The method comprises positioning the target portions of the rodents in the path of a radiation beam and simultaneously irradiating each target portion so that each target portion of each rodent receives about same amount of radiation dose while the non target portion of the rodent is protected from radiation within a rodent station, wherein the rodent stations are arranged about the periphery of the radiation beam approximately equidistance from the center of the radiation beam at a predetermined distance from the beam source. While the examples use mice and the stations are named rodent stations, the device and method disclosed herein are applicable to other small animals of similar size. During radiation treatment, rodent target portion is positioned within the periphery at a location closer to the center of the radiation beam than the shielding chambers and approximately equidistant from the center of the radiation beam. In some embodiments, the dose rate of radiation delivered to the targeted portions is within 5% of the expected dose rate. And the total dose delivery per fraction is within 5% of anticipated dose per fraction delivery. The rodent stations are housed in a sealed chamber anesthetic gas is delivered into the chamber to maintain anesthesia of the rodents contained therein. During treatment, at least three rodents are radiated simultaneously. In some embodiments, five rodents are radiated simultaneously. In some embodiments, the variance of the radiation experienced at the exposed target portion of each housed rodent is less than 30% of each other, in other embodiments, less than 20%, in additional embodiments, less than 15%, in further embodiments less than 10%, and in some embodiments, less than 5%.

The present application additionally relates to methods for rapid radiation treatment of a plurality of small animals with focal dosing. An example method comprises placing a plurality of rodent stations within the periphery of a radiation beam approximately equidistance from the center of the radiation beam such that each station receives approximately same amount of radiation. The small animals are housed in the station with target portions of the rodents exposed to the radiation beam while the non target portion of the rodents are protected by a shield chamber of the stations, wherein each target portion of the rodents receive approximately equal amount of clinically relevant radiation dose rate. In some embodiments, the small animals are mice with tumor grafts and the clinically relevant radiation dose rate is 250-400 cGy per min. In some embodiments, five mice are treated simultaneously. In some embodiments, the dose rate of radiation delivered to the targeted portions is within 5% of the expected dose rate. And the total dose delivery per fraction is within 5% of anticipated dose per fraction delivery. The variance of the radiation experienced by the rodent is less than 10% of each other. In some embodiments, the radiation is repeated a plurality of times for fractionated radiation treatment of the small animals. A radiation dose of at least 0.1 Gy, at least 0.2 Gy, at least 0.5 Gy, at least 1 Gy, at least 2 Gy, at least 5 Gy, at least 10 Gy, at least 15 Gy, at least 20 Gy, at least 25 Gy, at least 30 Gy, at least 35 Gy, at least 40 Gy, at least 45 Gy, at least 50 Gy, at least 100 Gy is delivered in at least at least 1 fraction, at least 2 fractions, at least 4 fractions, at least 6 fractions, at least 10 fractions, at least 15 fractions, at least 20 fractions, at least 25 fractions, at least 30 fractions, at least 40 fractions, at least 50 fractions using the radiation treatment device described herein depending on the condition and the size of the animal. In one embodiment, a total of 40-50 Gy is delivered in 20-25 fractions to each of the small animals using the radiation treatment device disclosed herein. The survival rate of the radiation treated small animals using the methods described herein is more than 80%, in some embodiments, more 85%, in other embodiments, more than 90%, in additional embodiments, more than 95%, and in further embodiments more than 96%. The amount of radiation received by the non-targeted portion of the animal is less than 20% of the target portion of the animal, in some embodiments, less than 15%, in other embodiments, less than 10%, in additional embodiments, less than 5%.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIGS. 1A-C are schematic drawings illustrating the radiation variance or uniformity among differently arranged rodent stations.

FIG. 1D is a schematic diagram providing a top view of a rodent ionizing radiation treatment device according to one embodiment of the present disclosure, showing five stations/units housed in a sealing chamber.

FIG. 1E is a side view illustration of a device built according to the design of FIG. 1D.

FIGS. 2A-F are illustrations of different components of an embodiment of the rodent ionizing radiation treatment device during different assembling stages.

FIGS. 3A-E are illustrations and diagrams showing the radiation measurements of the stations of the device using nanoDot dosimeters.

FIGS. 4A-F are illustrations showing the assembly of and the mice loading process into the rodent stations.

FIGS. 4G&H are illustrations of an alternative shield design for cranium exposure.

FIGS. 5A-D are illustrations of radiation measurements performed with differently arranged rodent stations.

FIGS. 6A&6B are Kaplan-Meier event-free survival (EFS) curves of the standard radiotherapy dosing treatment groups comparing non-irradiated mice group versus irradiated with implanted xenografts.

FIGS. 7A-D shows xenograft growth curves as a function over time in two rhabdomyosarcoma (RMS) lines in mice undergoing the standard radiotherapy dosing schedule versus untreated controls.

Like reference numbers and designations in the various drawings indicate like elements.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

The multi station/unit radiation device disclosed herein address unmet needs in radiation treatment related applications. The rodent ionizing radiation treatment device disclosed herein enable simultaneous radiation treatment of a plurality of small animals such as mice with localized radiation therapy. The device can provide clinically-relevant homogeneous radiation dosing and scheduling to a plurality of small animals or other in vivo cancer models simultaneously. Specifically, the multi station/unit design of the device allows for rapid loading of anesthetized small animals into radiation shield chambers that absorbs over 95% of the delivered dose and protects the remainder of the untreated animal, allowing for more clinically accurate recapitulation of radiotherapy regimens. The loaded chambers can then be placed in appropriate position, with tumor/target exposed and the non-targeted portions of the animals blocked/shielded. The device comprises a sealing chamber that houses the stations/units and has an inlet for anesthetic gas such as isoflurane containing gas or gas mixture to maintain the animals in the proper treatment position during radiation delivery. After treatment, the animals can be returned to their cages for recovery.

Although it is assumed that at a given level inside a radiation beam cone, the amount of radiation received inside the beam cone would be uniform/homogenous, it has been discovered however significant variation exists when shield chambers are placed at different location of the radiation field as shown in FIG. 1A or if multiple shield chambers are used, when the shield chambers are arranged randomly in the radiation field as shown in FIG. 1B. This discovery is aided by accurate reading of radiation using nanoDot dosimetry that revealed the heterogeneity of radiation received by various stations when multiple stations are used. It has been further discovered that by placing the stations uniformly inside the radiation field as, for example, illustrated in FIG. 1C, the stations received comparable amounts of radiation within acceptable variance and without significant decrease in dose rate received at each station. The device disclosed herein therefore is built around the concepts of uniformly distributed stations that enable homogenous radiation dose being applied to each station to enable high-throughput radiation treatment of small animals. The variance of radiation experienced at the exposed target portion of the animals in the stations in general is less than 30% of each other, although in some embodiments, the variance is less than 20% of each other, in further embodiments, less than 15%, in additional embodiments, less than 10%, in some embodiments, less than 5%. The device exemplified below comprises 5 stations. The device however is adaptable to embodiments where at least three stations are uniformly spaced that receive uniform radiation, in some embodiments four stations, in further embodiments 6 stations, and in additional embodiments 7-12 stations. A person of ordinary skill in the art will recognize that additional numbers of stations within the device that each received predetermined amount of radiation are contemplated and are within the present disclosure.

The device disclosed herein can be made compatible with commercially available irradiators such as the RS2000 Irradiator from Rad Source Technologies, Inc (Suwanee, Ga.), which is the most commonly purchased X-ray based irradiator used for research purposes. The RS 2000 X-Ray Biological Irradiator is used as an example to illustrate the concept and utility of the rodent station described herein. Although the design concepts discussed below are directed to the RS2000 irradiator, ionizing radiation treatment device compatible with other irradiators can be similarly designed based on the concepts discussed herein.

### Detailed Designs of Example Device

Example rodent ionizing radiation treatment devices described herein comprise a plurality of rodent stations/units uniformly placed about equidistance around a center piece to ensure homogenous radiation beam and dose distribution during a radiation treatment. A schematic illustration of an example embodiment of the device 100 is shown in FIG. 1D. The device 100 comprises five rodent stations/units 102, 104, 106, 108, and 110. The stations/units are substantially identical to each other and are uniformly assembled around a center piece 150 of the device to the bottom of a sealing chamber 160. Each station comprises components that are exemplified by the illustration and numbering of unit 104. Specifically, unit 104 comprises a shield chamber 112 with an open end 114 and a closed end 116, a base 118 with a clip 120, and an optional head cap 122 to cover the open end 114. The base of the station 118 optionally comprises a hole 126 that can be aligned with an slot 124 on the bottom of the sealing chamber to ensure accurate placement of the unit relative to the rest of the component of the device. Each unit of the device is shown to be fully assembled with the head cap. The bottom of the sealing chamber 160 comprises a marked circle 162 that will be aligned with marked circle for example shown in FIG. 3A inside a radiation device during a radiation treatment session. The sealing chamber 160 comprises an inlet 164 for the delivery of anesthesia gas or gas mixture into the sealing chamber. The sealing chamber 160 can comprise an optional handle 166 for opening up the lid portion of the chamber. FIG. 1E is a side view illustration of a device made according to the scheme of FIG. 1D. The side view in FIG. 1E reveals the lid portion 160a and the bottom 160b of the sealing chamber 160. The sealing chamber is shown to be made from poly(methyl methacrylate) material. Although other material transparent to radiation known in the art can be used as well. The handle 166 can employ a locking mechanism to ensure the lid portion 160a is sealed with the bottom 160b. The inlet 164 is shown to be connected to a gas line 168. The side view also reveals the longitudinal side view of the shield chamber 136, head cap 130 of shield chamber 128, and closed end 116 of shield chamber 112. The sealing chamber in general is optionally made of material that would not substantially block or absorb radiation.

Components of the rodent station 104 are shown in FIG. 2A. The station is shown to have the shield chamber 112 fixed on the base plate 118, which has the clip 120 and the hole 126. Beside the chamber and base is a shield 112s with aperture 112a and the head cap 122. The shield chamber 112 is shown to comprise a side opening 112o, the open end 114 and the closed end 116. FIG. 2B is a side view illustration of the assembled station 104, showing the closed end 116, the head cap 122 covering the open end of the shield chamber 112, the shield 112s covering the side opening 112o of the chamber 112, a nanoDot dosimeter 170 placed on top of bolus stack 172 that covers the aperture 112a. The dosimeter 170 is used to measure the amount of radiation received at the aperture 112a. During actual experiment, the small animal is placed inside the chamber with head covered with the head cap and the portion of body to be irradiated aligned with the aperture to be exposed to radiation. The components of the five rodent stations of the device are shown in FIG. 2C. While the stations in group “a” and the head caps in group “b” are shown to be approximately identical to each other, the shields in group “c” are shown to have variance from each other. Specifically, the apertures 112a1, 112a2, 112a3, and 112a4 are shown as circular collimations with diameters ranging from 3 mm to 30 mm. During a given treatment, when uniform radiation is to be applied to each animal, the shield pieces will have uniform shape and aperture size. In between the treatments, however, a different set of shield pieces with different uniform shape and aperture size may be used depending on the amount of radiation to be received by the animals. For example, during the course of tumor treatment, as the radiation therapy progress, the tumor size may initially swell followed by subsequent shrink. The shields used for the different treatment sessions would then have different size apertures to fully expose the tumor while protecting the healthy surrounding tissue, i.e. bigger sized aperture for swelled tumors and smaller sized aperture for shrunk tumors. In some embodiments, even if the tumor shrinks, the original sized aperture is used to ensure complete radiation treatment coverage of the initial tumor base.

The device at different stages of assembly is illustrated in FIGS. 2D-2F. Specifically, FIG. 2D shows the bottom 160b of the poly(methyl methacrylate) sealing chamber with the marked circle 162, the center piece 150, and the slots 124. The base and shield chamber pieces are then assembled along the marked circle 162 of the bottom 160b as shown in FIG. 2E. All five stations are shown to be fully assembled with the head and shield pieces as shown in FIG. 2F with dosimeters 170, 174, 176, 178, and 180 properly placed to measure radiation at the apertures of the stations. Although the embodiment exemplified in FIG. 2 comprises openings and open end at specific locations of the chamber, the opening and open end of the chamber can be designed to be at other location(s) of the chamber to allow easy loading of the animal while proper exposure of the portion of the animal to receive treatment. The orientation of the chamber can be adjusted as well. For example, if the brain portion of the animal is to be irradiated, with the head of the animal extended from the open end of the chamber, the shield chamber would not have the side opening so the body of the animal is shielded from the irradiation. The shape and size of the shield piece and its aperture can be adjusted accordingly to expose the brain portion of the animal that is to be irradiated while the rest of the head of the animal is protected with the shield piece. In some applications, it may be more suitable to have the shield chambers aligned longitudinally perpendicular to the periphery of the device so the brain portions of the animal are directed to the center piece of the device. In other applications, the brain portions can be directed to the periphery of the device away from the center depending on the location of the brain portion to be irradiated and the amount of radiation that is suitable for the treatment. Regardless of the orientation, shape, size, and location of the opening(s) of the shield chambers, the radiation beam is distributed evenly across the beam cone footprint, the stations being distributed evenly and symmetrically across the beam footprint, with the unshielded portion at approximately equal distance to the center of the footprint.

In one embodiment, the device is designed to be fit into a Rad Source RS2000 laboratory irradiator (Suwanee, Ga.). The sealing chamber of the device is optionally approximately 35×35×7 cm to fit inside Rad Source RS2000 laboratory irradiator. Accordingly, each shielding chamber of the device is about 4 inch in length and about 1.5 inch in diameter. The individual shielding units can be made of acrylic tubes covered in 1.63 mm lead foil. Each tube has a cut-out and platform as described above to allow an anesthetized mouse's body to be protected within the tube, while the left leg is extended immobilized, exposing the flank xenograft. The Lead head caps and additional foil with pre-cut circular collimation to create a 2-3 cm surface margin of beam exposure were then added to cover all unshielded areas. The individual units were shaped so that all five xenografts are aligned within the base of the beam cone, equidistant from the beam center, to ensure an approximate equal dose-rate.

Dosimetry Quality Assessment (QA) Using NanoDot Dosimeters

The decisive issue in biological radiation experiments is the radiation dosage, which indicates how much radiation dose that a target actually receives and how much it is intended to receive. An error in dose will give a bias result from the experiment. RS 2000 X-Ray Biological Irradiator is a radiation unit that generates spectral kV X-ray beams to irradiate cells or small animals in experiments. Its simplicity to operate and the much less radiation shielding materials required have made it popular and widely used for cell/small animal experiments. FIG. 3A is the inside view of an RS 2000 X-Ray Biological Irradiator (Suwanee, Ga.) showing circumferential circles at the base and vertical levels on the side while FIG. 3B illustrates the footprint of the radiation cone at any given level. Inside a given beam footprint, the radiation dose is believed to be uniformly distributed. When RS2000 X-Ray Irradiator is used to perform an experiment, the machine manufacturer provides a default method to calculate the dose delivered to the target which is simply given by multiplying the time of irradiation with the dose rate at certain position of the irradiator (level # or circle #, for instance) as shown in FIG. 3B with corresponding dose rate listed in Table 1 below.

TABLE 1

| | Field | Dose rate at center beam | |
|---|---|---|---|
| Level | Size (cm) | (filtered) cGy/min | Dose rate (raw) cGy/min |
| 7 | 8 | 1000 | |
| 6 | 12.1 | 800 | 3100 |
| 5 | 16.4 | 550 | 1750 |
| 4 | 20.6 | 330 | 1250 |
| 3 | 25.1 | 230 | 850 |
| 2 | 29.5 | 170 | 500 |
| 1 | 33.8 | 110 | 400 |

If the irradiated target is at the machine-defined dose calculation position, the dose received by the target should be close or equal to the calculated dose. However, the typical running energy of the irradiator is 160 kV with a mean energy of about 55 kV. This low energy X-ray is attenuated rapidly inside tissues, and the absorbed dose is quite sensitive to depth and position. If an experimenter uses a customized tray to hold cell tubes or small animals, as the target position likely to be off from the level and circle, the actual dose delivered to the target will not be the same as the one given by dose rate multiplying with time. Additionally, as a mouse has a certain size, the dose at different depth inside the mouse body will be different, and this difference is decided by the dose gradient. How to measure the actual dose delivered to the target and how large the dose gradient becomes a crucial issue. A simple and reliable measurement method is disclosed below to measure the dose and the dose gradient in the target during the cell/small animal experiments. Specifically, commercial nanoDot dosimeters are used to measure the dose delivered to the targets in the cell/small animal experiment that uses the RS 2000 X-Ray Irradiator.

LANDAUER (Glenwood, Ill.) InLight nanoDot dosimeters are used to perform the dose measurement for cell/small animal radiation experiments with RS 2000 X-Ray Biological Irradiator (Suwanee, Ga.). NanoDot dosimeter is a type of optically-stimulated luminescent detector (OSLD). The detectors are made of aluminum oxide crystals (Al₂O₃). When a dosimeter is irradiated with photon or charged particle beams, the electrons in the aluminum oxide crystals are excited to different energy levels. Using green light from a laser or light emitting diode source to stimulate the irradiated dosimeter, a blue light will emit from Al₂O₃ which is collected and amplified by a photo-multiplier tube and turned into electric current. The amount of blue light emitted is proportional to the dose of irradiation. LANDAUER MicroStar Reader is such a device to measure the dose that a nanoDot dosimeter gets irradiated.

Irradiator Calibration

In order to use nanoDot dosimeters to measure the radiation dose, the LANDAUER MicroStar Reader is calibrated by irradiating nanoDot dosimeters with a set of known doses at specific positions defined by the irradiator. For example, a group of five nanoDot dosimeters are placed on the Level 4 circle inside a RS2000 irradiator at the positions of 0, 72, 144, 216, and 288 degrees on the circle. The dose rates without the rodent stations present at these five positions are known by multiplying the dose rate with the time of irradiation. Each group of dosimeters is irradiated for 12 seconds (50.4 cGy), 24 seconds (100.8 cGy), 36 seconds (151.2 cGy), 48 seconds (201.6 cGy), 60 seconds (252 cGy), and 72 seconds (302.4 cGy) respectively. By correlating the readings of nanoDot dosimeters with the given irradiated doses, the dose-reading relationship for the irradiator under the specific running condition such as 160 kV 25 mA is established. The established calibration curve is validated by exposing arbitrary known doses to a set of nanoDot dosimeters, using the LANDAUER nanoDot Reader to measure the doses, and then compare the measured dose with the known number.

Rodent Station Radiation Dose Measurement

After the calibration process disclosed above, the radiation dose at each aperture of the rodent station is measured and recorded using the nanoDot dosimeters. Specifically, referring to FIG. 3D, nanoDot dosimeters 170, 174, 176, 178, and 180 are shown to be placed in the aperture of five rodent stations 104, 102, 110, 108, and 106 respectively. The device was then placed inside the RS 2000 irradiator with the circle 162 marked on the bottom 160b of the device 100 aligned with the circle 4 (labeled as 182) of the RS 2000 irradiator shown in FIG. 3A. The aligned device has nanoDot dosimeters placed at positions corresponds to 0, 72, 144, 216, and 288 degrees on the circle 4 of the irradiator. The device is then irradiated for 44 seconds for a target does of 210 cGy at 160 kV 25 mA and the dose measurement according to the nanoDot dosimeters at the five position points are listed in Table 2 below. As shown in Table 2, the amount of radiation received at the aperture of each rodent station are very similar to each other with a variance of 18.4 cGy between the lowest average dose at 288 degrees and the highest average dose at 144 degrees, demonstrating less than 10% radiation variation among the rodent stations. The average recorded doses at each stations 208.4 cGy, 215.8 cGy, 217.9 cGy, 204.0 cGy, and 199.5 cGy are very close to the targeted dose of 210 cGy.

TABLE 2

| Degrees on | | Readings | | |
|---|---|---|---|---|
| circle #4 | Sensitivity | (cGy) | Average Dose (cGy) | Error (cGy) |
| 0 | 0.82 | 209.068 | | |
| 0 | 0.82 | 208.804 | | |
| 0 | 0.82 | 208.382 | | |
| 0 | 0.82 | 207.474 | 208.4 | 0.3 |
| 72 | 0.80 | 217.766 | | |
| 72 | 0.80 | 210.665 | | |
| 72 | 0.80 | 217.707 | | |
| 72 | 0.80 | 217.076 | 215.8 | 1.5 |
| 144 | 0.79 | 218.502 | | |
| 144 | 0.79 | 217.855 | | |
| 144 | 0.79 | 218.001 | | |
| 144 | 0.79 | 217.162 | 217.9 | 0.2 |
| 216 | 0.81 | 203.902 | | |
| 216 | 0.81 | 203.861 | | |
| 216 | 0.81 | 204.279 | | |
| 216 | 0.81 | 203.943 | 204.0 | 0.1 |
| 288 | 0.82 | 200.032 | | |
| 288 | 0.82 | 198.980 | | |
| 288 | 0.82 | 199.282 | | |
| 288 | 0.82 | 199.563 | 199.5 | 0.2 |

Dose Gradient Measurement

To study the dose gradient of the X-ray inside the irradiated target (dose decrease/cm), nanoDot dosimeters are placed under different thicknesses of water-equivalent bolus and irradiated with X-ray at 160 kV 25 mA, the doses at different thicknesses are then measured and the dose variation over the thickness of bolus is calculated to determine the dose gradient. Specifically, as illustrated in FIG. 3C, several layers of 5 mm tissue equivalent bolus 172 was placed to recapitulate the presence of a xenograft-bearing mouse flank. NanoDots 170, 170′, and 170″ were placed at the top of bolus to capture the surface dose, in between the bolus pieces, as well as under the boluses respectively to determine the dose attenuation by depth within the animal model. Referring to FIG. 3E, boluses 172 and 172′ are shown to wrap the dosimeters as sandwiches with one of the rodent stations 104 enlarged. All five stations are used with the same arrangement shown in FIG. 3D, although with stacked bolus and nanoDots as specified in Table 3 below for each station. The data points in Table 3 corresponds to the nanoDot readings for each sandwich associated with a specific polar angle and three nanoDot dosimeters are placed on the top, in the middle, and at the bottom of each layer of bolus. The average of dose variation per cm is about −23% with a standard deviation of about 4%. This result indicates that the attenuation of x-ray beam from RS 2000 biological irradiator is significant and this effect should be taken into account when planning a mouse radiation experiment.

TABLE 3

| Set up | 0° | 72° | 144° | 216° | 288° |
|---|---|---|---|---|---|
| Top nanoDot (cGy) | 250.6 | 270.0 | 262.7 | 257.7 | 249.5 |
| Bolus (cm) | 0.5 | 1 | 0.5 | 1 | 0.5 |
| Middle nanoDot (cGy) | 225.9 | 205.5 | 225.5 | 207.2 | 214.2 |
| Bolus (cm) | 0.5 | 0.5 | 0.5 | 0.5 | 0.5 |
| Bottom nanoDot (cGy) | 191.7 | 197.9 | 198.6 | 175.9 | 197.4 |

Dose Measurement Under High Energy Beam

The nanoDot dosimeter is sensitive to radiation beam energy. Its responses to low energy beam and high energy beam are different in that it gets more attenuated for low energy beam (kV) than high energy beam (MV). Hence, generating a dose-reading calibration curve that is going to be used for cell/small animal experiments with RS 2000 Irradiator is under a certain beam condition. In this example, 160 kV 25 mA is used. If a dose-reading calibration curve is generated under different X-ray beam energy other than the RS 2000 X-ray beam but the calibration curve is used to perform the cell/small animal experiments, a correction factor is applied so that the measurement readout can give the true dose. Table 4 shows the comparison of the results using RS 2000 X-ray beam and Linac 6 MV beam for the calibrations. Both calibration methods give consistent nanoDot measurement results although they are not exactly the same (see Table 4).

TABLE 4

| | Dose (cGy) (RS 2000 | Dose (cGy) = reading × 0.48 | Diff |
|---|---|---|---|
| Sensitivity | beam calibration) | (MV beam calibration) | (%) |
| 0.96 | 216.99 | 441.75 × 0.48 | |
| 0.96 | 214.94 | 453.53 × 0.48 | |
| 0.96 | 216.23 | 421.48 × 0.48 | |
| 0.96 | 216.78 | 448.88 × 0.48 | |
| Average | 216.23 | 211.64 | 2.1 |

According to the manufacture, the accuracy of the nanoDot dosimeter dose measurement is 5%. This level of fluctuation had been observed in multiple time measurements for the same dosimeter. In general, the same dosimeter was measured multiple times (e.g. four times) and took the average. The fluctuation may come from the difference of positioning of the dosimeter in the nanoDot Reader when it is placed each time. The angular dependence, temperature dependence, and dose rate dependence are small for nanoDot dosimeters. Energy dependence is larger for low beam energy but when the energy is up to 1 MeV the energy dependence can be ignored. For a nanoDot dosimeter, the dose measurement range is from 10 mrad to 1500 rad. Compared with other types of dosimeters, such as films, diodes, or ion chambers, nanoDot dosimeter demonstrates better performance on many aspects, especially in the cell or small animal experiments. The measurement method described herein can be applied universally to perform in-vivo dosimetry for the cell/small animal experiments that use RS 2000 X-Ray Biological Irradiators or similar devices.

### High Throughput Radiation Treatment Protocol

The rodent ionizing radiation treatment device (RTD) described above can be used to radiate five mice simultaneously. The protocol used for the radiation treatment is described in detail below.

Pre-Irradiation Care:

Mice are received at 6-8 weeks of age and are allowed a 10-14 day acclimation period. Once or twice weekly during this time mice are offered supplemental nutrition in the form of diet gel or flavored nutritional supplements. This acclimates the mice to the novel food and assures that body weights remain stable during this period. At the end of the acclimation period, body weights are recorded for each mouse. This body weight is used to calculate an average starting weight for the group. Additionally, smaller mice may be separated out and allowed to gain weight prior to implantation. This is especially important in mice that are to be placed on outcomes studies as the irradiation procedure may cause mice to lose weight, so a starting weight of 17 grams or higher is preferred. In preparation for implantation, the left hind limb and left side of the mice 200 is shaved 1-3 days prior to the procedure as shown in FIG. 4A, exposing the tumor bearing flank 202.

Xenograft Implantation Procedure:

Xenograft implantation is performed under a biosafty cabinet. Xenograft tissue is cut into sections approximately 2×2 mm and kept in media until ready for implantation. Mice are placed in an anesthesia induction chamber and anesthetized with 5% isoflurane and 4 L per minute of O₂ until breathing has slowed and mice are unresponsive to a toe pinch. The flank area of the mice is wiped with ethanol. A small incision, approximately 4 mm, is made in the skin on the left flank with scissors. The scissors are then guided into the incision and advanced distally toward the patella, opening and closing the scissors a few times to loosen the skin from the underlying muscle and creating a pocket. Using forceps, a small piece of tumor tissue is inserted into the pocket. A drop of pen-strep is placed into the incision and the incision is closed by placing a small drop of tissue glue to the proximal edge of the incision and carefully lifting and touching the skin from the opposite edge to the drop of glue. Care is taken not to get the tissue glue into the incision. The mouse is placed back into a clean cage and observed for recovery. The cell line, passage number and date of implantation are recorded on the cage card. The xenograft growth and healing of the incision in the days following surgery of the mice are monitored. Incisions generally heal with no complications. Slower growing tumors can be monitored once or twice a week, and faster growing tumors more frequently. When tumors are noted to reach a size of ˜1×1 cm, mice are sorted into cages and assigned to treatment groups.

Irradiation Process:

On the first day of treatment, prior to irradiation, tumors are measured via calipers and the size is recorded. Body weights are measured and recorded. Under a sterile biosafty cabinet, mice are anesthetized with 5% isoflurane at 4 L per minute of O₂ in an anesthesia chamber. The mice are monitored until breathing has slowed to 2 seconds between breaths, after which they are quickly removed one by one and placed into rodent stations before being placed into the RTD and maintained there with 5% isoflurane at 4 L per minute of O₂. FIG. 4B is a illustration of a dissembled rodent station 104 showing the lead sheet covered chamber 112 fixed on the base plate 118 with a side opening 112o, which has the clip 120 and the hole 126. Beside the chamber and base is the lead shield 112s with the aperture 112a and the head cap 112. The anesthetized mice 200 is quickly placed inside the chamber 112 with the tumor bearing leg 214 secured with clip 120 as shown in FIG. 4C. The chamber 112 is then covered with shield 112s and cap 122 with the tumor bearing flank 202 exposed through the aperture 112a. The loading process is repeated with the other four rodent stations 102, 106, 108, and 110 and the mice loaded stations are then assembled into the RTD 100 as shown in FIG. 4E with the lid of the sealing chamber closed to form a sealed chamber 160. The loaded RTD 100 is shown to be placed inside a sealing chamber 160 that is connected to anesthesia gas through inlet 164. The anesthesia is maintained for ˜30-60 seconds to assure the mice are sufficiently anesthetized before the RTD 100 is moved to the RS 2000 irradiator 230, shown in FIG. 4F. The platform 232 inside the irradiator is placed at the fourth level and the RTD 100 is transferred to the irradiator 230 and immediately connected to the isoflurane supply 234 within the irradiator. The marked circle 162 on the bottom of the RTD 100 is aligned with the 4th circle on the plate of the irradiator (shown as 182 in FIG. 3A) and mice are irradiated for a time as determined by the desired delivered dose and calibrated dose-rate at the flank. After irradiation anesthesia is turned off and mice are returned to the bio-safety cabinet and immediately removed from the shielding and RTC and placed back in the home cage to recover from anesthesia. In embodiments when brain tumor is treated, FIG. 4G shows an alternative shield design that protects the head of the mice while providing exposure of tumor bearing cranium. Specifically, shield 190 is shown to cover the head of the mice while exposing the tumor bearing cranium portion 204 of the head. The head of the mouse is secured with clip 192. FIG. 4H is a side view of the alternative shield design. Although not specifically shown in FIGS. 4G and 4H, during the radiation treatment, the only portion of the mice that is to be exposed is the targeted portion, for example, the tumor bearing cranium 204, while the rest of the body of the animal is shield from radiation. As discussed above related to the design illustrated in FIG. 2, the heads of the mice in some embodiment are aligned perpendicular to the periphery of the device, towards the center piece to allow more direct radiation beam exposure than the periphery arrangements.

Post-Treatment Animal Care:

At the beginning of treatment, mice are provided with supportive care as the irradiation process can lead to weight loss, skin lesions and general failure to thrive. Side effects from irradiation usually begin to appear after the second week of therapy, and can last for 4 to 6 weeks. It is critical to monitor and provide supplemental care during this period to ensure mice survive this period. Nutritional supplementation are provided to mice on treatment in the form of lab diet gel and/or flavored commercially available irradiated mouse treats to support appetite and help maintain body weight. If mice refuse to eat these, moistened lab blocks may be provided. Sub Q fluid therapy are provided preemptively and as needed to prevent and correct dehydration and help support over all body condition. These treatments were given at least weekly during irradiation treatments and continued after treatment based on weekly body weight and hydration status for as long as needed. If body weight drops below 16 grams mice will be monitored closely and may be euthanized if this condition is coupled with lethargy, loss of body temperature or other abnormal condition or behavior, and does not resolve or respond to the supportive care described in the section. After 3-4 weeks from the start of irradiation treatment, lesions will appear on the treated flank area which may be painful depending on the severity. Rimadyl® infused flavored nutritional supplements (Rodent MD's™ available through BioServe (Beltsville, Md.)) may be offered during this period, alternately, Rimadyl® may be administered subcutaneously at 5 mg/kg for 3-7 days until lesions heal.

Xenograft Harvesting:

Mice are euthanized prior to xenograft tissue harvesting by placing in a CO₂ chamber and death are verified via cervical dislocation. The skin over the tumor is removed with a forceps and scalpel while care is taken not to contaminate the xenograft tissue with the external hair and skin. Once the tumor is exposed it is removed from the underlying flank muscle tissue using a scalpel blade and dropped into a clean dish. From there it is divided into multiple sections for analysis and stored appropriately.

Efficacy and Toxicity Assessment:

Skin toxicity is measured via a scoring system. Skin reaction usually will develop between the second and third weeks after the start of irradiation treatments. Mice are observed for skin reaction changes 3× week for 43 days beginning on the 15th day from the beginning of irradiation and given a score of 0-5 based on the following grading system:
0 no visible skin reaction
1 faint erythema and/or faint dry desquamation
2 patchy dry desquamation
3 confluent dry desquamation
4 patchy moist desquamation
5 confluent moist desquamation

Efficacy is determined by weekly tracking of tumor size and response for 16 weeks following the end of treatment. The formula to determine volume is ((widest xenograft measurement, mm/2+perpendicular measurement, mm/2)̂3*0.5236)1000=volume, cc. Event-free survival time is determined from the first treatment day until animals reach early removal criteria, die from other causes or reach the limit of observation time.

The response of each mouse to treatment is recorded including a weekly measurement of the tumor beginning on the first day of treatment and ending the 16th week following the completion of treatment. Complete response and recurrence events are also recorded during this period and an overall treatment failure rate are reported at the end of the 16 week observation period.

Dose Measurement at Different Body Parts

The nanoDot dosimeters are used to study the doses different body parts of a mouse were exposed to during a typical radiation treatment in a rodent station and the results are listed in Table 5 below, showing less than 10% radiation is experienced in non-exposed body parts of the mice as compared to the exposed tumor bearing flank. The rodent station is effective at providing protection for the mice while exposing sufficient radiation to the tumor.

TABLE 5

| | | | | | | Dose |
|---|---|---|---|---|---|---|
| Body part | Rd1 | Rd2 | Rd3 | Rd4 | Avg. | (cGy) |
| under chin | 19.151 | 18.470 | 19.03314 | 19.54707 | 19.0504 | 9.13 |
| under belly | 43.34736 | 41.72878 | 42.53216 | 41.74059 | 42.3372 | 20.30 |
| on top of back | 34.16947 | 34.39985 | 33.5433 | 34.96301 | 34.2689 | 16.43 |
| on flank | 513.9401 | 499.7371 | 479.0106 | 498.4336 | 497.7804 | 238.66 |

### EXAMPLES

RS 2000 X-ray Biological Irradiator (Rad Source Technologies, Inc., Suwanee, Ga.) is used to irradiate the mice in the follow examples. The machine was run at 160 KV and 25 mA with its standard 0.3 mm of Cu filtration. The X-ray generated under this condition had an energy spectrum with the minimum energy of 45 KV up to maximum 160 KV, and the half value of the beam was 0.62 mm of Cu. The dose gradient of this X-ray in tissue-equivalent bolus was about −10% per 0.5 cm depth. Quality assurance of the shielding and xenograft dosing was performed using nanoDot dosimeters (Landauer Inc. Glenwood, Ill.). Dose readings at xenograft surfaces and with layered bolus phantoms were used to calculate the appropriate “beam-on” treatment times. Dosimeters placed at the throat, back, and under the abdomen of animals undergoing treatment were compared to the doses received by the xenograft (unshielded) to determine the percent blockage of non-targeted animal by the device. These dosimeters were calibrated under the beam conditions described above.

### Example 1

### Radiation Quality Assessment of the Rodent Stations

Radiation doses within the #4 circle with all 5 rodent stations present (fully loaded device) were evaluated. To assess the amount of radiation experienced by mice at different anatomy locations, all five stations were loaded with mice and with leg tumor exposed to radiation. As shown in FIG. 5A, the rodent stations were placed at the center of the circle, at 12 o'clock, at 3 o'clock, at 6 o'clock, and at 9 o'clock. The average radiation in the #4 circle was 201 cGy. The radiation dose throughout the fully loaded device in #4 cone was 2 Gy/fraction. For comparison, when only one rodent station was present, the radiation at the center of the circle is 204 cGy, about 12% less than when all five stations were present. The amount of radiation experienced by the mice at tumor, throat, back and belly in each rodent station were recorded. As comparison, the amount of radiation experienced by the mice at tumor, throat, back and belly when covered by the radiation stations without the shield piece were tested and recorded. The data for center beam and 12 o'clock position are listed and compared in the Table 6 below.

TABLE 6

| | Center Beam (cGy) | | | 12 o'clock (cGy) | | |
|---|---|---|---|---|---|---|
| | with | without | % | with | without | % |
| | shield | shield | change | shield | shield | change |
| Tumor | 231 | 235 | −2 | 211 | 218 | −3 |
| Throat | 11 | 22 | −50 | 9 | 13 | −30 |
| Back | 5 | 17 | −71 | 5 | 18 | −72 |
| Belly | 11 | 35 | −71 | 9 | 126 | −93 |

As shown by the data in Table 6, the rodent station with shield piece does not reduce more than 5% of the radiation dose experienced by the tumors compared to the rodent station without shield piece. The shield on the other hand, significantly reduces the radiation doses experienced at non-target mice anatomy including throat, back, and belly. The dosing of the rodent station with shield also appears to be more consistent when compared to the rodent station without shield: for example, the dosing experienced at the belly of the mice without the shield at center beam is only 35 cGy compared to 126 cGy at the 12 o'clock.

Dose Variance by Location Studies

Dose heterogeneity has been observed at different locations of a radiation beam in a given circle when rodent stations were tested. Specifically, when the stations were positioned between center and periphery as shown in FIG. 5B, radiation dose was shown to vary by as much as 40 cGy, which equals to about 20% of the target dose. When the stations were randomly arranged as shown in FIG. 5C, radiation dose was shown to vary by as much as 38 cGy, which equals to about 19% of the target dose. The standard deviation among the various randomly arranged stations were shown to be 11.1 cGy. When the stations were in circular arrangement as shown in FIG. 5D, the apertures of the stations were approximately equidistance from the center, represented by the dotted line in the figure. Radiation dose was shown to vary by 7 cGy, which equals to about 3.5% of the target dose. The standard deviation among the various evenly arranged stations were shown to be 3.4 cGy. Significant improvement in radiation homogeneity has been observed in uniformly positioned rodent stations.

### Example 2

The Pediatric Preclinical Testing Program (PPTP) has been successfully used to determine the efficacy of novel agents against solid tumors by testing them within a mouse-flank in vivo model. The present example examines the feasibility and biologic outcomes of radiation therapy applied to xenograft lines from the PPTP program using alveolar and embryonal rhabdomyosarcoma xenograft lines.

Two rhabdomyosarcoma xenograft lines from the PPTP, Rh30 (alveolar) and Rh18 (embryonal) were selected. Using established methods, xenografts were implanted, grown to appropriate volumes, and were subjected to fractionated radiotherapy. Tumor response-rates, growth kinetics, and event-free survival time were measured. In the present study, the rate of acute toxicity requiring early removal from study in 93 mice was only 3%. It was observed that the alveolar Rh30 xenograft line demonstrated a significantly greater radiation resistance than embryonal Rh18 in vivo. This finding was validated within the standardized 30 Gy treatment phase, resulting in overall treatment failure rates of 10% versus 60% for the embryonal versus alveolar subtype, respectively. The study demonstrated the rodent ionizing radiation treatment device described herein enables safe, clinically-relevant focal radiation delivery to immune-compromised mice. It further recapitulated the expected clinical radiobiology.

The Pediatric Preclinical Testing Program (PPTP) is a well-established initiative that employs panels of pediatric solid tumor and leukemia xenograft models that recapitulate the clinical experience with commonly administered chemotherapeutic agents in a variety of childhood malignancies. It, therefore, currently serves as a valuable model to test the efficacy of novel agents, and has a strong record of correctly predicting the responses of various malignancy types to standard and novel therapies that are observed in the clinical setting. Seventy-five percent of PPTP models have been derived from direct transplant of tumor tissue into mice, hence tissue culture artifacts have been avoided. Some reports have suggested that these procedures preserve tumor initiating cells when tumor fragments are directly implanted within the flanks of severe combined immunodeficient (SCID) mice. The influence of tumor/tissue environment is preserved when using xenografts (which include stromal and tumor initiating cells of the original tumor). Xenograft models are thus favored over in vitro conditions for robust drug efficacy screening, due to these better recapitulated “real-world” phenomena. For these reasons the histologic as well as molecular phenotypes of the PPTP disease models remain preserved after multiple passages within these mice.

Given the robust recapitulation of the clinical biology demonstrated by the panels of tumor xenografts, applying radiation enables a comprehensive investigation of mechanisms of radiation resistance across a wide spectrum of pediatric malignancies. Similar to the improved drug efficacy prediction with in vivo models (versus in vitro testing), radiotherapy has been shown to follow suit. Incorporating radiotherapy into candidate drug screening therefore provides an extra dimension to this process to allow the identification of potential synergism and/or enhanced toxicity within a living system.

Groups of five mice were anesthetized at once in an induction chamber with continuous 5% isoflurane and 4 L/min O₂. The treatment device received this same anesthetic gas mixture and flow-rate during mouse loading and treatment as well. Anesthetized mice were removed one at a time from the induction chamber and placed in their individual units with shielding added as described in the protocol above. Five mice would then undergo a single 2 Gy fraction and subsequently were removed from anesthesia and allowed to recover in warm conditions. From induction to recovery the mean treatment time was 10 minutes.

Xenograft Lines and Mice:

The xenograft lines used were two rhabdomyosarcoma (RMS) lines: Rh18 (embryonal) and Rh30 (alveolar), which were harvested at diagnosis and engrafted into mice prior to the donor patient having received any cytotoxic therapy. These lines were created directly from patient samples propagated in mice-only and were never grown in vitro. These two lines express distinct genotypes; Rh30 expresses the fusion transcription factor PAX3-FKHR (present in 70% of alveolar RMS) while Rh18 does not. This distinction was confirmed via RT-PCR for the fusion product. After implantation, xenografts were allowed to grow until reaching approximately 0.5-1.0 cubic centimeter in volume and were then randomized to treatment or control (no treatment) groups; with the first measurement representing Day 0 of experiments.

CB17SC scid^{-/-} female mice (Taconic Farms, Germantown N.Y.), were used to propagate subcutaneous tumors. All mice were maintained under barrier conditions and experiments were conducted using protocols and conditions approved by the institutional animal care and use committee of The Ohio State University. Female mice were used irrespective of the gender from which the tumor was derived. Tumor volumes (cm³) were determined weekly, to determine growth and response, as described by Houghton et al. in Pediatr Blood Cancer 2007:49(7):928-940, entitled “The pediatric preclinical testing program: description of models and early testing results”, incorporated herein by reference.

Metrics, Endpoints and Statistics:

In addition to tumor growth kinetics, the complete response (CR) rates, recurrence rates and 12-week failure rates were ascertained. A CR was defined as a complete xenograft disappearance during/after treatment. A recurrence was defined as a measureable xenograft reappearing after at least one week of CR and followed by at least two weeks of growth. A 12-week treatment failure was either the lack of complete response or recurrence within the 12-week post-treatment observation period. Animal event-free survival was ascertained and defined from the time of treatment initiation (or beginning of observation for controls) until the time at which animals were removed from the study for the following events: 1) when xenografts reached four times their relative volume [final tumor volume/initial volume]; 2) the absolute volume exceeded 2.5 cm³; and/or 3) the xenograft grew to severely impede mobility or comfort of the subject mouse. Treatment-related events were defined as animals which failed to complete the study due to excessive treatment toxicity which warranted early removal.

Differences in event-free survival times among groups were compared using the log-rank method. Actuarial complete response rates, recurrence rates and 12-week treatment failure rates between groups were compared using the Fisher's exact test. The differences between the means of groups were compared using a Student's t-test. Additionally, since a range of radiation doses and initial xenograft volumes were used, a unit of the given radiation dose divided by volume (cm³) of the initial xenograft size (dose-density, Gy/cm³) was used to compare for any dose-volume differences that may have affected outcomes. This is based on the radiobiologic principle that the likelihood of complete tumor eradication, for a given dose, is partially dependent on the number of cells present at the inception of treatment.

Shield Aperture Physics Quality Assurance:

The ionizing radiation treatment device (RTD) described herein made it possible to irradiate five mice at a time while minimizing prohibitive side effects from daily radiation and anesthesia exposure. To estimate the minimum doses to the base of the xenograft, a 0.5 cm bolus was used to approximate the implanted tumor size at the initiation of treatment. The measured dose at 0.5 cm depth of tissue equivalent bolus, with 1.0 cm circular collimation (most frequently used size) was 202 cGy [range 193-221±7 cGy] at an approximate dose-rate of 285 cGy/minute. With 2.5 cm circular collimation (for the largest initial tumors), the measured dose was 221 cGy [range 214-26±7 cGy]. These slightly higher doses were deemed acceptable, given that in the clinic the maximum point dose is often higher than the target dose due to practice of prescribing to isodose lines to encompass volumes (with maximum point doses often between 7%-12% higher than the prescribed dose). Measurements of doses received by the shielded, non-targeted animal, relative to the unshielded xenograft doses were 4.2% [±0.6%] under the abdomen, 2.0% [±0.3%] and 2.2% [±0.2%] for the back, representing 95.8-98.0% shielding for all anatomic sites.

Results from Dosing Phase

A dose of 30 Gy in 15 fractions over 3 weeks was given to groups of mice bearing the Rh30 and Rh18 xenografts while separate, untreated groups represented the controls (10 mice per group) using the protocol outlined above. Kaplan-Meier event-free survival (EFS) curves of the standard radiotherapy dosing treatment groups were plotted in FIG. 6. FIG. 6A shows EFS curves of non-irradiated mice 602 versus irradiated 604 with implanted xenografts. The median EFS was 15 versus 75 days for untreated versus treated mice, respectively (p<0.001). FIG. 6B shows event-free survival radiated versus non-irradiated mice, stratified by xenograft line. There was no significant difference between the untreated groups (Rh18 and Rh30 controls, 606 and 608, respectively). There was a trend in improved EFS in the treated Rh18 group 610 versus the treated Rh30 group 612, p=0.072; log-rank. The median survival of Rh18 group could not be calculated due to the lack of events within the 12-week observational period. The addition of radiotherapy significantly improved the overall event-free survival rate from 17 days to 106 days, p<0.001, log-rank. There was a trend in improved event-free survival between the Rh18 versus the Rh30 group (log-rank, p=0.072; the median could not be determined for the Rh18 group due to only 1 event occurring during the observation period.) No significant difference was noted between the mean dose densities of the Rh30- and Rh18-bearing mice groups in the standard treatment phase as well (80 Gy/cm³ versus 87 Gy/cm³, respectively.) Xenograft growth curves as a function over time in two rhabdomyosarcoma (RMS) lines in mice undergoing the standard radiotherapy dosing schedule versus untreated controls are shown in FIG. 7. Rh18 (embryonal RMS) untreated growth is shown in FIG. 7A compared to irradiated Rh18 growth shown in FIG. 7B demonstrating, one 12-week recurrence. The Rh30 (alveolar RMS) untreated growth is shown in FIG. 7C compared to irradiated Rh30 growth shown in FIG. 7D, demonstrating seven 12-week recurrences. There were no acute treatment-related deaths. Both groups demonstrated a 10/10 complete response rate. There was one recurrence within the Rh18 group and 6/10 recurrences in the Rh30 group, resulting in a 12-week treatment failure rate of 1/10 versus 6/10 (for Rh18 and Rh30 groups, respectively, p=0.029, Fisher's exact).

By incorporating radiotherapy into robust in vivo models, like the PPTP, relevant radio-resistance mechanisms and their predictive biomarkers, as well as the comprehensive preclinical testing within the context of novel agents can be studied and discovered. Two xenograft lines representing the two most common subtypes of pediatric RMS: alveolar (ARMS; Rh30) and embryonal (ERMS; Rh18) were selected and used. The former is the most biologically-aggressive subtype of this disease, confers a five-year overall survival of less than 50% (10-30% when metastatic at presentation), and requires higher doses of radiation to be delivered across all stages and surgical groupings when compared to the more favorable ERMS, which confers a 73% five-year overall survival. Despite progress made in this disease, control of the primary site is still a major source of treatment failure, as ⅔ of the relapses observed in Intergroup RMS Study-IV (n=888) were local-regional, suggesting an inherent resistance to radiotherapy in this disease. The studies disclosed herein recapitulate this clinical experience. The ARMS line, harboring the characteristic PAX3:FKHR translocation was clearly the more resistant than the ERMS line. This example demonstrates that radiotherapy can be effectively applied to the PPTP system and reliably recapitulates the clinical experience with external beam radiotherapy using the rodent radiation device that provides reproducible, daily conventional fractionation in a high-through put format. The approach described herein can be applied to the studies of other malignancies of the PPTP as well.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A rodent ionizing radiation treatment device, comprising a plurality of rodent stations each including a shielding chamber that houses and shields a portion of a rodent from a projected ionizing radiation beam while leaving a target portion of each housed rodent exposed to the projected radiation beam such that the exposed target portion of each housed rodent is positioned within the radiation beam approximately equidistant from the center of the projected radiation beam at a predetermined distance from the beam source, wherein each exposed target portion of the rodent receives about same amount of radiation, with a homogeneous beam and dose distribution.

2. The device of claim 1, wherein the plurality of rodent stations are contained in a sealable chamber.

3. The device of claim 2, wherein the sealable chamber comprises an inlet for delivery of anesthetic gas and oxygen mixture into the chamber when sealed.

4. The device of claim 1, wherein the one or more shielding chamber comprises lead of sufficient thickness to substantially reduce or eliminate irradiation dosing of the shielded portion of animal housed in the chamber.

5. The device of claim 1, wherein each shielding chamber is positioned about the periphery of the projected radiation beam.

6. The device of claim 5, wherein the shielding chambers deflect and scatter radiation from the projected radiation beam to enable homogeneous beam quality and radiation dose being applied to the stations.

7. The device of claim 1, wherein there are three, four, five, six, seven, eight, nine, ten or more rodent stations.

8. The device of claim 1, wherein there are between 4 and 8 rodent stations.

9. The device of claim 1, wherein there are five rodent stations.

10. The device of claim 1, wherein each station comprises a base with a clip to stabilize the rodent.

11. The device of claim 1, wherein each station comprises a shield to cover an opening of the shielding chamber.

12. The device of claim 11, wherein each shield comprises an aperture that exposes the target portion of each housed rodent to receive about same amount of radiation during treatment.

13. The device of claim 1, wherein each shield chamber comprises an open end and the station comprises a head cap to provide shield to the open end of the chamber.

14. The device of claim 13, wherein the open end of each chamber is covered with a shield with aperture that receives about same amount of radiation dose during treatment.

15. The device of claim 1, wherein the longitudinal dimension of the chambers are positioned about the periphery of the projected radiation beam.

16. The device of claim 14, wherein the longitudinal dimension of the chambers are positioned perpendicularly to the periphery of the projected radiation beam and the open end of each chamber is directed inside the periphery towards the center of the beam.

17. The device of claim 1, wherein the device is sized to be placed in a commercially available ionizing radiation treatment device.

18. The device of claim 1, wherein the variance of the radiation experienced at the exposed target portion of each housed rodent is less than 10% of each other.

19. A method of irradiating target portions of a plurality of rodents simultaneously, comprising positioning the target portions of the rodents in the path of a radiation beam and simultaneously irradiating each target portion so that each target portion of each rodent receives about same amount of radiation dose while the non target portion of the rodent is protected from radiation within a rodent station, wherein the rodent stations are arranged about the periphery of the radiation beam approximately equidistance from the center of the radiation beam at a predetermined distance from the beam source.

20. The method of claim 19, wherein the rodent stations deflect and scatter radiation from the radiation beam to enable homogeneous radiation and doses being applied to each rodent target.

21. The method of claim 19, wherein each rodent target portion is positioned within the periphery at a location closer to the center of the radiation beam than the shielding chambers and approximately equidistant from the center of the radiation beam.

22. The method of claim 19, wherein the dose rate of radiation delivered to the targeted portions is within 5% of the expected dose rate.

23. The method of claim 19, wherein the total dose delivery per fraction is within 5% of anticipated dose per fraction delivery.

24. The method of claim 19, wherein the rodent stations are housed in a sealed chamber that comprises an inlet for supply of gas into the sealable chamber.

25. The method of claim 24, further comprising delivering gas into the chamber through the inlet wherein the gas anesthetizes or maintains anesthesia of the rodents contained therein.

26. The method of claim 19, wherein the rodents are anesthetized before being positioned inside each rodent station.

27. The method of claim 19, wherein each rodent station comprises a base with a clip and the target portion of the rodent to be radiated is stabilized with the clip.

28. The method of claim 19, wherein each station comprises a shield with an aperture to cover an opening of chamber wherein the target portion of the rodent is exposed to the aperture and each aperture receives about same amount of radiation.

29. The method of claim 19, wherein the chambers are arranged longitudinally about the periphery of the radiation beam and the target portion of the rodent is the leg of the animal.

30. The method of claim 19, wherein each chamber comprises an open end and the rodents are positioned inside the chamber with heads extending through the open ends of the chambers wherein the head of the rodents is covered with a shield piece with an aperture that exposing rodent cranium to receive radiation.

31. The method of claim 30, wherein the chambers are arranged longitudinally perpendicular to the periphery of the radiation beam and the rodent cranium are directed inside the periphery towards the center of the beam to receive radiation that are incident to the rodent cranium in a perpendicular manner.

32. The method of claim 19, wherein the radiation is performed in a commercially available ionizing radiation treatment device.

33. The method of claim 19, wherein at least three rodents are radiated simultaneously.

34. The method of claim 19, wherein five rodents are radiated simultaneously.

35. The method of claim 19, wherein the variance of the radiation experienced by rodents is less than 10% of each other.

36. A method for rapid radiation treatment of a plurality of small animals with focal dosing, comprising placing a plurality of rodent stations within the periphery of a radiation beam approximately equidistance from the center of the radiation beam such that each station receives approximately same amount of radiation, wherein the small animals are housed in the station with target portions of the rodents exposed to the radiation beam while the non target portion of the rodents are protected by shield chambers of the stations, wherein each target portion of the rodents receive approximately equal amount of clinically relevant radiation dose rate.

37. The method of claim 36, wherein small animal is mice with tumor graft and the clinically relevant radiation dose rate is 250-400 cGy per min.

38. The method of claim 37, wherein five mice are treated simultaneously.

39. The method of claim 36, wherein the dose rate of radiation delivered to the targeted portions is within 5% of the expected dose rate.

40. The method of claim 36, wherein the total dose delivery per fraction is within 5% of anticipated dose per fraction delivery.

41. The method of claim 36, wherein the variance of the radiation experienced by the rodent is less than 10% of each other.

42. The method of claim 36, wherein the radiation is repeated a plurality of times for fractionated radiation treatment of the small animals.

43. The method of claim 42, wherein a total of 40-50 Gy is delivered in 20-25 fractions to each of the small animals.

44. The method of claim 43, wherein the survival rate of the radiation treated small animals is more than 95%.

45. The method of claim 36, wherein the amount of radiation received by the non-targeted portion of the animal is less than 5% of the target portion of the animal.
